(19) United States
(12) Patent Application Publication
(10) Pub. No.: US 2003/0120302 A1
(43) Pub. Date: Jun. 26, 2003

(54) Vaso-occlusive device with serpentine shape

(75) Inventors: John Laurence Minck JR., Fremont, CA (US); Kevin M. Jaeger, Fremont, CA (US); Jacqueline Tu Tran, Milpitas, CA (US)

Correspondence Address: DAVID T BURSE, BINGHAM MCCUTCHEN LLP, THREE EMBARCADERO CENTER, SUITE 1800, SAN FRANCISCO, CA, 94111-4067, US

(73) Assignee: SCIMED Life Systems, Inc.

(21) Appl. No.: 10/027,080

(22) Filed: Dec. 20, 2001

(51) Int. Cl. A61M 29/00

(52) U.S. Cl. 606/200

(57) ABSTRACT

A vaso-occlusive device having a length, at least a portion of the length having a serpentine secondary shape when the device is in a relaxed condition. The vaso-occlusive device deliverable to a vasculature site such that as the device is deployed, it immediately forms along the wall of the vasculature site, thus occluding the site.

### BACKGROUND OF THE INVENTION

1. Field of the Invention

The field of the invention is vaso-occlusive devices.

2. Background

Vaso-occlusion devices are surgical implements that are placed within vessels, typically via a catheter, to block the flow of blood through the vessel making up that portion of the vasculature or within an aneurysm stemming from a vessel. One commonly used vaso-occlusive device is a helical wire coil having windings that are dimensioned to engage the wall of an aneurysm. In treating aneurysms, it is common to place multiple such coils within the aneurysm. The coils occlude the site by posing a physical barrier to blood flow and by promoting thrombus formation at the site. The sites are accessed with flexible, relatively small diameter catheters, such as those shown in U.S. Pat. Nos. 4,739,768 and 4,813,934. Once the site has been reached, one or more coils are placed into the proximal open end of the catheter and advanced through the catheter with a pusher. When the coil(s) reach the distal end of the catheter, they are released into the vessel site by the pusher into the vessel.

Prior art vaso-occlusive coils generally have a linear shape when in a tensioned condition, i.e., stretched or compressed, and a folded or convoluted shape when in an untensioned or relaxed condition. A stretched or compressed condition allows the coil to be pushed through a catheter to the desired site in the vessel. As the coil is pushed out of the distal end of the catheter, it assumes its relaxed, i.e., folded or convoluted, shape, which is better suited for occluding the vessel. A variety of relaxed shapes have been employed in vaso-occlusive devices, such as those shown in U.S. Pat. Nos. 6,024,765, 6,254,592, and 4,994,069.

Notably, as each coil is released from the catheter at the vessel site, the distal end of the coil tends to move or “float” within the vessel structure, until a sufficient length of the coil has been released. Only after a sufficient length of the coil has been released from the catheter, does the coil lodge in the vasculature structure to form an occlusion. Because of this tendency to float, the coils tend to compartmentalize as they are released from the catheter. When a coil compartmentalizes, the entire length of the coil released from the catheter lodges in only a portion of vessel, preventing the coil from adequately occluding the vessel site. This floating and compartmentalization make placement of the coil in the desired vessel location, such as at an aneurysm, more difficult.

Additionally, linear coils, as they are deployed from a catheter, require a certain amount of breaking force to make the coil bend or fold. This breaking force also pushes the coil against the wall of the aneurysm, which could result in the rupture of the aneurysm if too much force is applied.

### SUMMARY OF THE INVENTION

One aspect of the invention is directed toward providing or employing a vaso-occlusive device having a length, at least a portion of the length having a serpentine shape when the device is in a relaxed condition, such that the vaso-occlusive device forms along the surface of a vessel as it is deployed, without significant floating or compartmentalization.

Other and further aspects and features of the invention will be evident from reading the following detailed description of the drawings, which is intended to illustrate, but not limit, the invention.

### BRIEF DESCRIPTION OF THE DRAWINGS

The drawings illustrate the design and utility of preferred embodiments of the present invention, in which similar elements are referred to by common reference numerals.

FIG. 1 is a side-elevational view of an embodiment of a serpentine-shaped coil in a relaxed or untensioned condition.

FIG. 2 is a side-elevational view of an embodiment of a coil with a serpentine-shaped distal portion and a substantially linear proximal portion, with the coil shown in a relaxed or untensioned condition.

FIG. 3 is an enlarged side-elevational view of an embodiment of a distal end of the serpentine-shaped coils of FIGS. 1 and 2.

FIG. 4 is an enlarged side-elevational view of a proximal portion of the serpentine shaped coils of FIGS. 1 and 2.

FIGS. 5A, 5B and 5C are side-elevational, partially cross-sectional views of an embodiment of a serpentine-shaped coil being deployed from a catheter into an aneurysm.

### DETAILED DESCRIPTION OF THE PREFERRED EMBODIMENTS

Preferred embodiments of the invention will now be described in the context of helical coil devices. Such devices may be made of a metal, polymer or other material without departing from the inventive concepts taught herein. Further, it will be appreciated by those skilled in the art that other types of occlusive devices besides helical coils are contemplated by the invention, e.g., a flat wire or polymer strand, a bead-and-chain, or other primary shape, so long as it generally has a length when in a tensioned (i.e., stretched or compressed) condition, such as when it is being delivered through a catheter to a vesel location in a body.

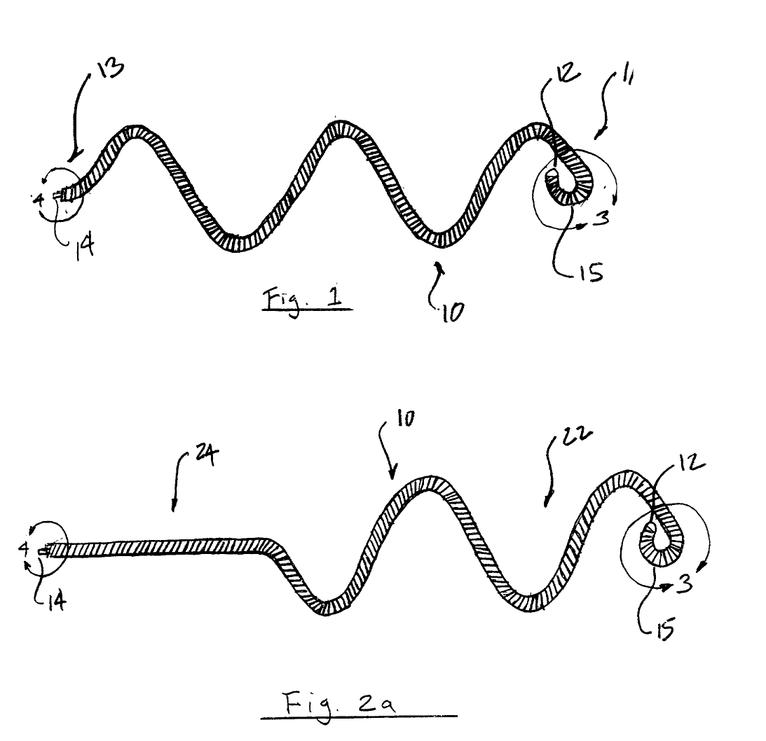

In a first preferred embodiment, a vaso-occlusive device 10 comprises a member 16 having a helical (i.e., coil) primary shape, and referred to herein as coil 16. When in a relaxed condition, the coil 16 has a generally serpentine secondary shape along its length, as shown in FIG. 1. The coil 16, in a relaxed condition, is free from external forces, namely compression and tension forces. In a constrained condition, such as in the lumen of a delivery catheter, the coil 16 will either be in a compressed condition or tensioned in a stretched condition, such that the coil 16 will assume a generally linear shape.

The coil 16 preferably is sufficiently small so that it may be advanced through the lumen of a catheter that is appropriately sized for accessing the targeted vascular site, such as an aneurysm. Notably, the coil 16 may be delivered to the vascular site by other delivery devices, which may allow for the coil 16 to be somewhat larger in dimension, but it must still be small enough to fit within the lumen of the vasculature at the delivery site. Although the vaso-occlusive device 10 will generally be described in conjunction with embolizing an aneurysm, it may also be adaptable for endovascular occlusion in arteries, veins, vascular malformations, and arteriovenous fistulas.

The serpentine secondary shape of the coil 16 has upper curves 20 and lower curves 21 occurring at substantially regular intervals over the length of the coil 16. The amplitude 18 of the serpentine secondary shape is the distance between the upper curve 20 and the lower curve 21. Preferably, the amplitude of the serpentine shape is between about 5 to 30 millimeters. The length of the coil 16 is preferably at least 15 times its amplitude when in its relaxed condition. Additionally, the coil 16 is preferably sufficient resilient so as to not deform out of its primary coil shape when stretched or compressed in a delivery apparatus.

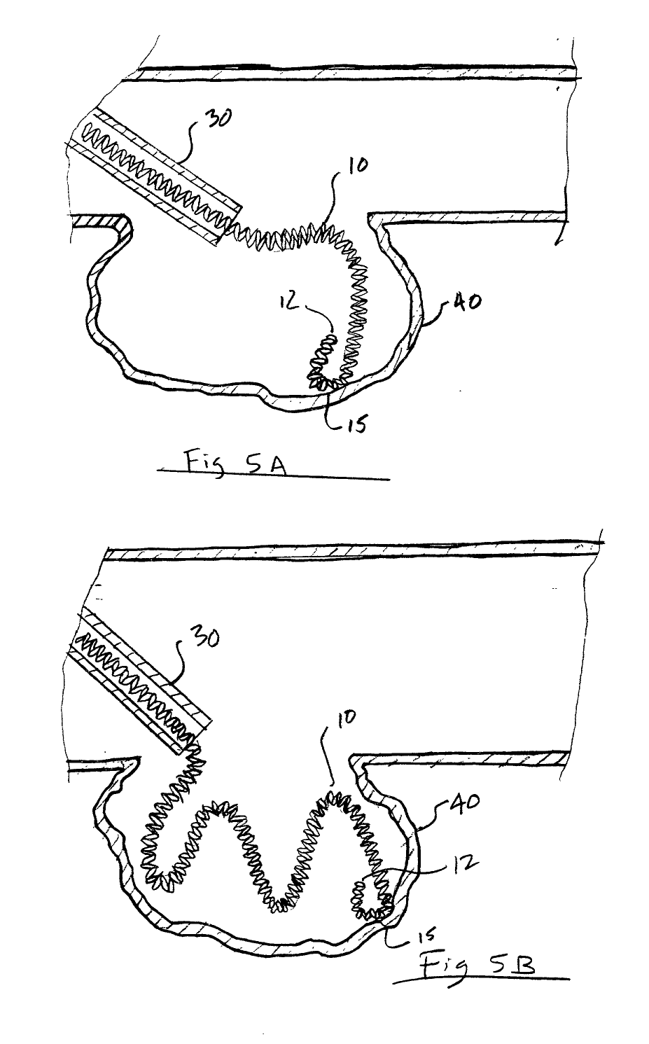

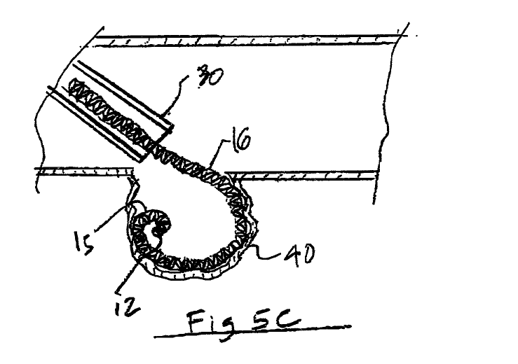

The coil 16 is desirably made up of a physiologically compatible, radiopaque material that may be viewed under fluoroscopy. Exemplary materials for the coil include platinum, gold, tungsten, or alloys thereof. However, the coil 16 could also be a polymer with radiopaque marker material added to the coil 16. Preferably, the coil 16 has a shape memory, such that, as the coil 16 is pushed out of a delivery catheter lumen it will naturally assume its relaxed, serpentine secondary shape. The deployment of the coil 16 will be discussed in more detail in conjunction with FIGS. 5A, 5B and 5C.

As discussed above, coils having a standard secondary shape typically require a significant amount of the coil to be deployed from the delivery catheter before the coil will lodge in the vascular site. This causes the coil to float or move within the blood stream of the vasculature structure as it is released. This floating or movement of the coil also may result in compartmentalization of the coil. When the coil compartmentalizes, the entire coil is deployed prior to the coil lodging in the aneurysm. The coil will then move or float until it lodges in only a portion of the aneurysm, resulting in inadequate occlusion of the aneurysm. The serpentine shape of the coil 16 as shown in FIG. 1 is preferable because as the coil 16 is deployed into the aneurysm, as shown in FIGS. 5A, 5B and 5C, the serpentine secondary shape allows the coil 16 to immediately form along the aneurysm as it is deployed. This allows the coil to be more easily, accurately, and predictably placed and helps to assure effective embolization of the aneurysm.

Further, linear coils, as they are deployed from a catheter, require a certain amount of breaking force to cause them to bend or fold. This force also pushes the coil against the wall of the aneurysm, which could result in the rupture of the aneurysm if too much force is applied to bend the coil. The serpentine secondary shaped coil 16 avoids the need for applying this breaking force to the coil because of its shape. As the coil 16 is deployed, it assumes its serpentine secondary shape, causing the coil 16 to bend on its own and form along the wall of the aneurysm without the need to apply an additional breaking force to the coil 16. Thus, the aneurysm is not subjected to the breaking force, which greatly reduces the possibility of the aneurysm rupturing as the coil 16 is deployed.

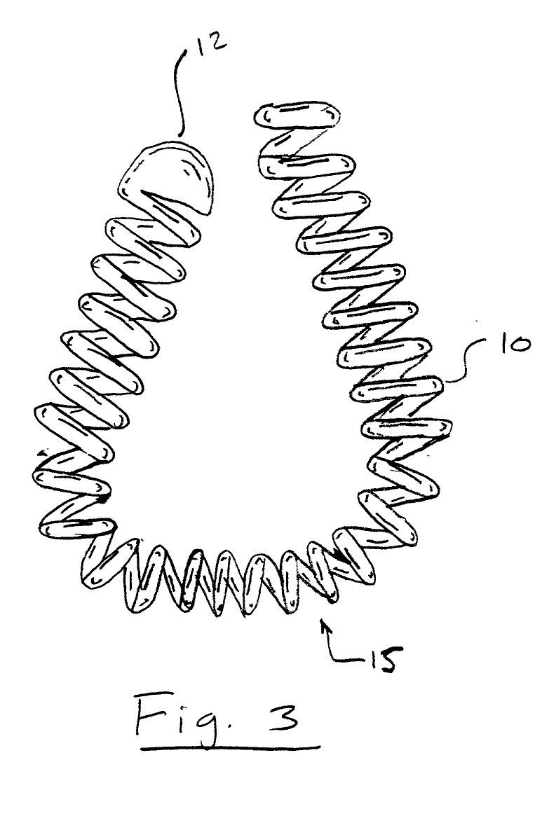

A distal end 11 of the coil 16 has a blunt, round, cap-like end 12, as shown in FIG. 3. The end 12 of the coil 16 is rounded to prevent the coil 16 from penetrating the weakened wall of the aneurysm when the coil 10 is delivered to the site. Additionally, the distal end 11 of the coil 16 is formed into a “J-shape” or loop 15. As the coil 16 forms along the wall of aneurysm, the J-shape or loop 15 at the distal end of the coil 16 prevents the tip 12 of the coil from puncturing the wall of the aneurysm. For smaller coils, the “J-shape” or loop 15 at the end of the coil 16 has a diameter of approximately between 2-5 cm. For larger coils, this diameter is approximately 10 cm.

A proximal end 13 of the coil 16 may be attached through an electrolytically erodible joint 14 to an insulated pusher wire 17. A direct current may be applied to the pusher wire 17. The current path, is in part, through joint 14 into the ionic medium surrounding the coil 16 upon deployment. The joint 14 erodes and allows the coil 16 to remain in the aneurysm. Although the coil 16 is shown and described as being electrolytically deployable, in other embodiments, the coil 16 may be deployed via other mechanisms such as a mechanical deployment mechanism.

In the first preferred embodiment, with substantially all of the length of the coil 16 having a serpentine secondary shape, it is preferable to use coils of a relatively short length. The shorter length allows the coil 16 to more easily be passed through a delivery catheter without the coil 16 deforming. In one embodiment, wherein substantially all of the length of the coil 16 has a serpentine secondary shape, the coil 16 is under approximately 40 cm in length.

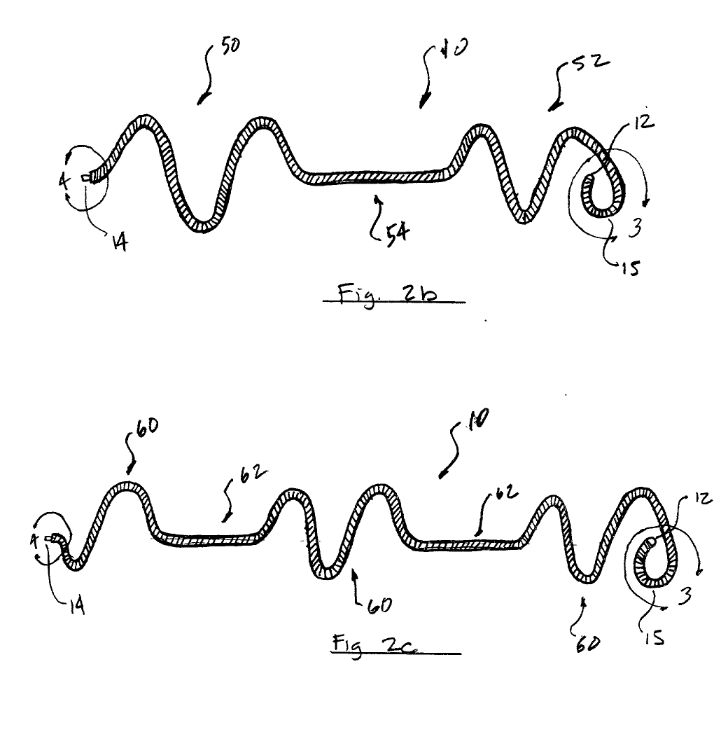

In a second preferred embodiment, as shown in FIG. 2a, the coil 16 has a serpentine secondary shape only in a distal portion 22. A proximal portion 24 of the coil 16 has a non-serpentine shape. In the embodiment shown, the proximal portion 24 of the coil 16 is substantially linear. A distal portion 22 of the coil 16 has a serpentine secondary shape to allow the coil 16 to form along the aneurysm as it is deployed from the catheter, as shown in FIGS. 5A, 5B and 5C, without moving or compartmentalizing. With only the distal portion 22 of the coil 16 having the serpentine secondary shape, the coil 16 may have a longer length than a fully serpentine coil, without deforming when pushed through a delivery catheter.

In a third embodiment, as shown in FIG. 2b, the coil 16 has a serpentine secondary shape on both a proximal portion 50, and a distal portion 52, with a linear middle portion 54. In yet a fourth embodiment, as shown in FIG. 2c, the coil 16 can alternate between a serpentine secondary shaped section 60 and a linear section 62 along the length of the coil 16. These embodiments also allow for the placement of longer coils, as in the second embodiment.

When a relatively long, stretch resistant coil is necessary, a coil 16 having a serpentine secondary shape along its entire length may be too difficult to push through the catheter without damaging the coil or the catheter, or without the coil being lodged (stuck) in the catheter. By having a serpentine secondary shape along only a portion of the coil 16, as shown in FIGS. 2a-2c, the coil 16 may be longer without these same problems, and also may more immediately form along the wall of a vessel deployment site.

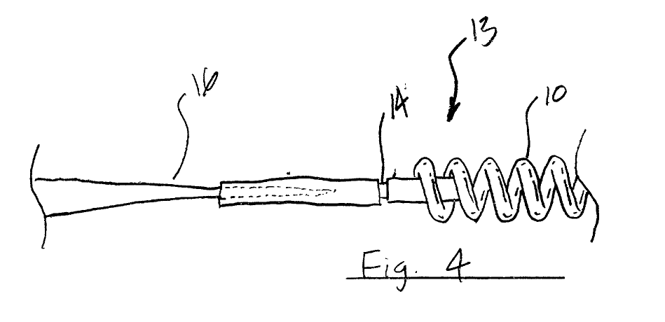

The distal portion of the each of the coils shown in FIGS. 2a-2c 10 preferably have blunt, round tips 12, as shown in FIG. 3. The distal portions additionally are formed into “J-shapes” or loops 15, as in the first embodiment. The proximal portions also are preferably attached to a pusher wire 17 through an electrolytically erodible joint 14, as shown in FIG. 4. The serpentine shaped portion of each coil preferably has an amplitude 18 between 5 to 30 millimeters.

In the preferred embodiments, the coils may be covered with a polymer, as described in U.S. Pat. No. 6,280,457, which is hereby incorporated by reference. The polymer further enhances cellular attachment and growth while maintaining favorable handling, deployment and visualization characteristics. Alternately, the coils may have a plurality of fibers attached along the length of the coils, as described in U.S. Pat. No. 5,304,194, which is hereby incorporated by reference. These fibers further enhance the ability of the coil to occlude the site by enhancing cellular attachment and growth.

With reference to FIGS. 5A, 5B and 5C, an exemplary method of deploying the vaso-occlusive device 10 of the first embodiment into an aneurysm 40 will now be described. As indicated above, while the method of deploying the coil 16 is described in conjunction with embolizing an aneurysm, the coil 16 may also be used for endovascular occlusion, by way of non-limiting examples, in arteries, veins, vascular malformations, and arteriovenous fistulas.

In one embodiment, the delivery apparatus is a catheter 30 positioned such that its distal end is at the mouth of the aneurysm 40, although other delivery devices are also possible. The coil 16, in its constrained condition within the delivery catheter lumen, will take on a substantially linear secondary shape. As the coil 16 is pushed out of the catheter 30, as in FIG. 5A, the substantially “J-shaped” end 15 is pushed against the wall of the aneurysm 40, but does not penetrate the wall. The distal end 11 of the coil 16 immediately forms along the wall of the aneurysm 40. In the case of a relatively small aneurysm, where the amplitude 18 of the coil 16 is larger than the interior space in the aneurysm 40, the coil 16 will attempt to assume its serpentine secondary shape. However, because the aneurysm 40 is smaller than the amplitude 18, the coil 16 cannot fully do so. This causes the coil 16 to form along and conform to the wall of the aneurysm 40 as it attempts to assume its serpentine secondary shape. The coil 16 will line the inner wall of the aneurysm, as shown in FIG. 5C, thereby forming an occlusion.

In the case of a relatively large aneurysm 40, where the amplitude 18 of the serpentine portion of the coil 16 is smaller than the interior space in the aneurysm, as shown in FIGS. 5A and 5B, the coil 16 will be able to fully assume its serpentine secondary shape. In this case, the coil 16 will immediately form along the wall of the aneurysm as it assumes its serpentine secondary shape, as shown in FIG. 5B.

Whether a relatively small or large aneurysm, as the coil 16 is further pushed out of the catheter 30, it continues to form along the wall of the aneurysm 40 and baskets the aneurysm 40, occluding it. Once the entire coil 16 is deployed, it is detached from the catheter 30, e.g., by sending an electrical current through the electrolytically erodible joint 14, eroding the joint and leaving the coil 16 in place at the site of the aneurysm 40.

As with the coil 16 of the first embodiment, each of the coils in the other embodiments assume their serpentine secondary shapes at their distal portions as they are deployed from the catheter 30. However, because in these embodiments only a portion of the length of each coil has a serpentine secondary shape, a longer coil may be deployed to the aneurysm 40.

As noted above, more than one coil 16 may deployed into the aneurysm 40 to occlude the site. Thus, the above-described exemplary method of deployment of the coil 16 may be repeated as necessary until the site is sufficiently occluded.

Thus, although several preferred embodiments have been shown and described, it would be apparent to those skilled in the art that many changes and modifications may be made thereunto without the departing from the scope of the invention, which is defined by the following claims and their equivalents.

What is claimed is:

1. A vaso-occlusive device, comprising:
a member having a length, at least a portion of the length having a serpentine shape when the member is in a relaxed condition.

2. The vaso-occlusive device of claim 1, wherein the member comprises a coil.

3. The vaso-occlusive device of claim 1, wherein substantially all of the length of the member has a serpentine shape when the member is in a relaxed condition.

4. The vaso-occlusive device of claim 1, wherein a distal portion of the member has a serpentine shape when the member is in a relaxed condition.

5. The vaso-occlusive device of claim 1, the member having a proximal portion, a middle portion and a distal portion, wherein the proximal portion and the distal portion have a serpentine shape, and the middle portion is a linear shape, respectively, when the member is in a relaxed condition.

6. The vaso-occlusive device of claim 1, wherein a proximal end of the member is electrolytically detachable from a delivery device.

7. The vaso-occlusive device of claim 1, wherein the serpentine shape comprises an amplitude of about 5-30 millimeters.

8. The vaso-occlusive device of claim 1, wherein the member, when tensioned in a stretched condition, has a length at least 15 times an amplitude of the serpentine shape.

9. The vaso-occlusive device of claim 1, wherein the member has a distal end having a substantially J-shaped tip.

10. The vaso-occlusive device of claim 1, further comprising a plurality of fibers fixedly attached to the member.

11. The vaso-occlusive device of claim 1, further comprising a polymeric fiber substantially covering the member.

12. The vaso-occlusive device of claim 11, wherein the polymeric fiber is wrapped around and onto a circumferential surface of the member.

13. The vaso-occlusive device of claim 1, wherein the member is stretch-resistant.

14. A method of occluding a selected site in a vessel with a vaso-occlusive device having a length, at least a portion of the length having a serpentine shape when the member is in a relaxed condition, the method comprising:
accessing the site with a delivery apparatus;
deploying the vaso-occlusive device from the delivery apparatus into the selected site of the vessel in a manner allowing a portion of the vaso-occlusive device to substantially assume its relaxed serpentine shape and form along a surface of the vessel at the site.
